(19) United States
(12) Patent Application Publication
(10) Pub. No.: US 2010/0255042 A1
(43) Pub. Date: Oct. 7, 2010

(54) Process for Producing Particles Loaded with Growth Factors as Well as the Particles Thus Obtained

(75) Inventors: Herbert Jennissen, Koeln (DE); Kristin Zurlinden, Haltern Am See (DE)

Correspondence Address: CAHN & SAMUELS LLP, 1100 17th STREET NW, SUITE 401, WASHINGTON, DC, 20036, US

(73) Assignee: Herbert JENNISSEN, Koeln (DE)

(21) Appl. No.: 12/740,007

(22) PCT Filed: Oct. 29, 2008
(86) PCT No.: PCT/EP2008/064677

§ 371 Date: Apr. 27, 2010

(30) Foreign Application Priority Data
Oct. 29, 2007 (DE) 10 2007 051 914.3

(51) Int. Cl. A61K 38/18; A61P 19/08; A61K 9/14

(52) U.S. Cl. 424/400; 514/8.8

(57) ABSTRACT

The present invention refers to a process for preparing a particulate material (particles) being loaded with growth factors, the thus obtained particles and the use for improving the ingrowth of implant material into the bone substance.

The present invention refers to a process for producing particulate material (particles) loaded with growth factors, the particles thus obtained and their use for improving the growth of implant materials into the bone substance, in particular for metallic or ceramic materials, which are used for implants such as artificial bones, joints, dental implants as well as micro implants.

The implantation of artificial joints or bones gained an increasing importance during the last years, for example for treating joint dysplasias or luxations as well as for diseases being based on the wear of joints due to joint malpositions. The function of implants and the materials being used for preparation thereof, which comprise metals such as titan or metal alloys as well as ceramics or plastic materials such as Teflon or polylactides, have been constantly improved so that implants can provide service lifetimes in 90 to 95% of the cases of up to 10 years after a successful healing process.

In spite of these advancements and improved surgical processes, an implantation is still a difficult and stressing intervention, in particular, as such implantation is connected with a long lasting healing process for the implant, comprising long lasting stays in clinics and health resorts including rehabilitation measures. Besides the pains, the length of the treatment as well as the isolation from the familiar environment are heavy stresses for the involved patients. Moreover, the long lasting healing process causes high costs for service and cure due to the required intensive care.

The understanding of the processes on the molecular level, being required for a successful in-growth of an implant, has been increasingly extended over the last years. Structure compatibility as well as surface compatibility are decisive for the tissue compatibility of the implant. The biocompatibility in a narrower sense is solely conditional on the surface. Proteins play an important role for all levels of integration. As discussed later, they decide already during the implantation surgery about the further process of the implant in-growth due to formation of an initial adsorbing protein layer, as the first cells settle on such layer.

For the molecular interaction between the implant, also named as biomaterial, and the tissue, a plurality of reactions takes places which appear to be extremely hierarchically structured. As a first biologically reaction, the adsorption of proteins on the surface of the biomaterial takes places. In the protein layer thus formed, single protein molecules are subsequently converted, for example, by conformational changes to signalling substances being presented on the surface, or protein fragments are delivered as signalling substances (molecular cues) by catalytic (proteolytic) reactions.

Triggered by the molecular cues, the cellular settling takes place in the next phase, which comprises a plurality of cells like leukocytes, macrophages, immunocytes and finally also tissue cells (fibroblasts, fibrocysts, osteoblasts, osteocytes). In this phase, further signalling substances, so-called mediators such as cytokines, chemokines, morphogenes, tissue hormones and real hormones play an important role. In the case of a biocompatibility, an integration of the implant into the complete organism takes place and ideally, a permanent implant is obtained.

In view of investigations which having made during the last years on the molecular level of osteogenesis, chemical signalling substances, the so-called “bone morphogenic proteins” (BMP-1-BMP-15) having an influence on the bone growth, gained an increasing importance. BMPs (in particular BMP-2 and BMP-4, BMP-5, BMP-6, BMP-7) are osteoinductive proteins, stimulating bone formation and bone healing by effecting proliferation and differentiation of the precursor cells to osteoblasts. Moreover, they help develop the formation of alkaline phosphatase, hormone receptors, bone specific substances such as collagen type 1, osteocalcine, osteopontine and finally the mineralisation.

The BMP-molecules regulate the three key reactions chemotaxis, mitosis and differentiation of the respective precursor cell. Moreover, BMPs play an important role in the embryogenesis, organogenesis of bones and other tissues, whereby osteoblasts, chondroblasts, myoblasts and vascular smooth muscle cells are known as target cells (blocking of proliferation by BMP-2).

Meanwhile, 15 BMPs including multiple isoforms are known. Except BMP-1, all BMPs are part of the “transforming growth factor beta” (TGF-13)-super family for which specific receptors on the surfaces of the respective cells have been found. As it could have been shown by the successful use of recombinant BMP-2 and/or BMP-7 in experiments for defect healing processes for rats, dogs, rabbits and monkeys, no specificity for any species seems to be present.

In the state of art, a number of experiments on the field of loaded materials and particles being used for promoting the growth of the bone substance, are known. Reports about the bondings of BMP-2 to hydroxyl apatite (HAP) go back to the beginnings of the BMP-research when it was found by Urist in 1984 that BMP can be chromatographically purified on a hydroxyl apatite column. In the same year already, Urist described an aggregate of BMP and TCP which induces the formation of cartilage in mice (U.S. Pat. No. 4,596,574). In the subsequent 20 years, a plurality of reports about the use of a combination of calcium phosphates (hydroxyl apatite, tricalciumphosphate) with BMP-2 has issued. Amongst others it was mentioned that BMP-2 is mixed with a defined amount of collagen or hydroxyl apatite and then, the mixture is immediately lyophilised and used after lyophilisation. In a further report, the adsorption of denaturised rh-BMP-2 in the presence of the denaturation agents such as urea to hydroxyl apatite has been studied. Even under such drastic conditions, only small amounts BMP-2 is bonded to hydroxyl apatite.

At present, BMP-2 will be therapeutically applied either as Induct Os® (Wyeth) on an “absorbable collagen sponge”, or in the form of Ossigraft® (Stryker). It is common to those materials that the concentration of BMP used per volume unit is relatively low, i.e. the required volume for ˜2 ml particle or sponge resp. for 1 mg BMP-2. Only under non-physiological conditions such as extreme ph-values in alkaline or acid ranges or in presence of detergents in a neutral range, larger amounts of BMP-2 are soluble. These amounts are in many cases insufficient for an application of BMP-2, being adapted to the size of the wound, and for an optimum stimulation of the bone growth, in particular in the presence of additional bone replacement substances. It is interfering that BMP-2 is provided to the organism by these application forms, due to an insufficient binding to collagen, at the same time in a single early delivery phase (“Burst phase”).

The invention described below is based on the observation that by adsorbing BMP-2 on a particulate material, in particular inorganic bone replacement material such as hydroxyl apatite, tricalciumphosphate, calcium carbonate, aluminum oxide or mixtures thereof, in particular bi- or triphasic mixtures thereof, an increased amount of BMP, in particular BMP-2 on the solid phase can be obtained per volume part compared to the above-mentioned materials, if the adsorption step is carried out for a sufficiently long period and at a controlled ph-value. After the adsorption step, a second step is preferably carried out as an extensive washing with at least 10-times liquid volume compared to the used solid phase volume. Hereby, it can be guaranteed that the amount of soluble BMP-2 in the liquid phase is removed. Thereby, a significant reduction of the so-called Burst-Phase to 1-2% of the adsorbed BMP-2-amount can be achieved. Thus, the option of applying BMP-2 in a high dose in a small compartment can be achieved. The coating of the surface in aqueous buffer solution can either be carried out in an acidic range in the range of pH 4 to 5, in particular at pH 4.5 or in a weakly alkaline range between pH 9 and 11, preferred at pH 10. Furthermore, it is of advantage if the particular material is a bioresorbable material.

By the inventive process, an increased amount of bone growth factor which cannot be washed off from the particles, is adsorbed to the surface, by means of adsorption as a form of chemical bonding which has to be distinguished from:
Mixing/combining with HAP or TCP (=mixture),
Including/entrapping in pores, for example,
Incorporation by, for example, lyophilisation of the liquid and precipitation in the material,
Coating of metals or ceramics according where particles or moulded bodies, for example, are immersed into a BMP-solution and immediately subjected to a drying step for removing the solution [the BMP-2 is dried as a layer on the surface (=no adsorption but adhesion)]

In bindings studies for BMP-2 (table 1) to various hydroxyl apatites, it was found by the inventors that BMPs, in particular BMP-2, can be linearly bound to calcium phosphate over a wide range in large amounts.

TABLE 1
Adsorption of BMP-2 at (pH 4.5) and desorption of BMP-2 (at pH 7.4) for
various particulate bone replacement materials

| | | | | | Bonit ® | | | |
|---|---|---|---|---|---|---|---|---|
| | Algisorb ® | | | | (13% SiO₂, | | | |
| | (80% TCP, | | Algipore ® | | 52% HAP, | | NuOss ® | |
| BMP-2 used in the | 20% HAP) | | (98% HAP) | | 35% TCP) | | (HAP, bovine) | |
| Incubation test | Control | APS | Control | APS | Control | APS | Control | APS |

Adsorption of BMP-2, mg/g

| | | | | | | | | |
|---|---|---|---|---|---|---|---|---|
| 0.1 | 0.53 | 0.78 | 0.58 | 0.81 | 0.26 | 0.23 | 0.45 | 0.69 |
| 0.2 | 1.06 | 1.54 | 1.27 | 1.52 | 0.52 | 0.39 | 0.61 | 1.16 |
| 0.3 | 1.35 | 2.14 | 1.52 | 2.27 | 0.67 | 0.52 | 0.98 | 1.41 |

Desorption, t_{1/2}, days

| | | | | | | |
|---|---|---|---|---|---|---|
| Half-life | 20.4 | 31.6 | 10.1 | 5.4 | 28.2 | 30.8 |

In Table 1, the results of adsorption experiments at pH 4.5 (20 mM Na-acetate pH 4.5) are shown, wherein the adsorption/incubation has been carried out over a period of at least 30 minutes (t_{1/2}=16 d), preferably over at least 1-2 hours (t_{1/2}=19 d) and, particularly preferred, for at least 4-6 hours (t_{1/2}=20 d). The longest half life values have been observed for incubations for 15-17 hours (t_{1/2}=23 d). The coating in the alkaline range can be preferably carried out in the presence of detergents such as SDS (buffer: 125 mM borate/0.066% SDS. pH 10.0). After adsorption step, the detergents are removed by 5-times washing in 10-fold material volume with PBS-buffer pH 7.4 (137 mM NaCl, 8.1 mM Na₂HPO₄, 2.7 mM KCl, 1.5 mM KH₂PO₄).

After the adsorption of BMP-2 on the particulate material, the desorption is measured. Thus, the samples are transferred each into 2 ml buffer ((50 mM Tris/HCl, 150 mM NaCl, pH 7.4). After predetermine intervals, the samples are taken out, washed in 3×2 ml buffer (50 mM Tris/HCl, 150 mM NaCl, pH 7.4) and counted in the γ-counter. Then, they are transferred into 2 ml fresh buffer for the next release interval. The amount of immobilized BMP-2 is determined by using ¹²⁵Iodine radioactive marked protein and counting in the γ-counter.

All bone replacement materials were incubated for 15 hours at room temperature with a predetermined concentration of BMP-2 in 20 mM Na-acetate-buffer pH 4.5. The desorption was determined in 50 mM Tris/HCl, 150 mM NaCl, pH 7.4. The half life times of the release in the so-called “Burst Phase”, which concern only 1-2% of the adsorbed amount of BMP-2, was between 0.4-1.1 days (not shown). APS: Aminopropyl triethoxysilane; Algipore® (density ˜0.63 g/cm³; ˜1.3×10⁴ particle/g) and Algisorb® (Dichte ˜0.63 g/cm³; ˜1.3×10⁴ particles/g), Co. Algoss GmbH, Wien; Bonit® Company DOT GmbH, Rostock; NuOss® Collagen Matrix ACE Surgical Supply Co. (Brockton, Mass., USA).

The invention will be further illustrated by means of the attached drawings. Thereby:

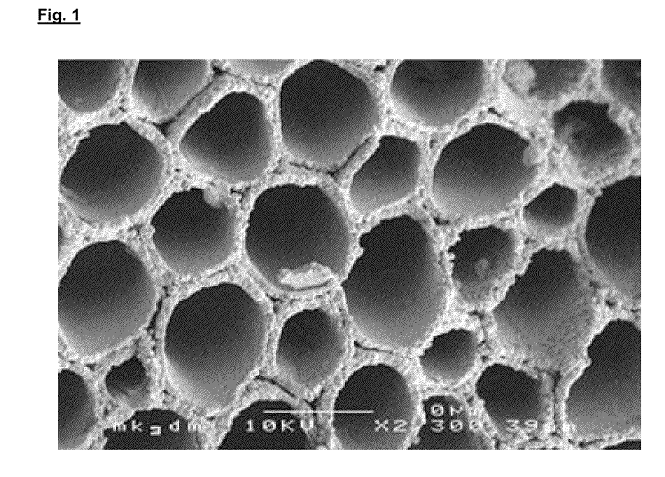

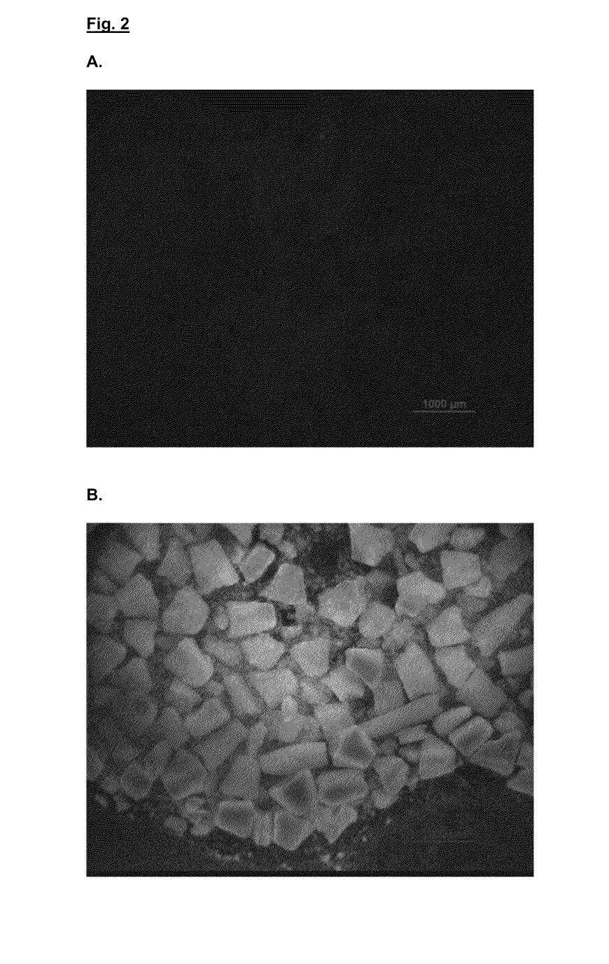

FIG. 1 shows the ultrastructure of a Algipore®-particle obtained from limestone algae (the unit corresponds to 10 μm. Algisorb® has the same structure. (taken from “Bone Augmentation in Oral Implantology”, Khoury, F. et al., page 349, 2007, Quintessenz Verlags-GmbH, Berlin);

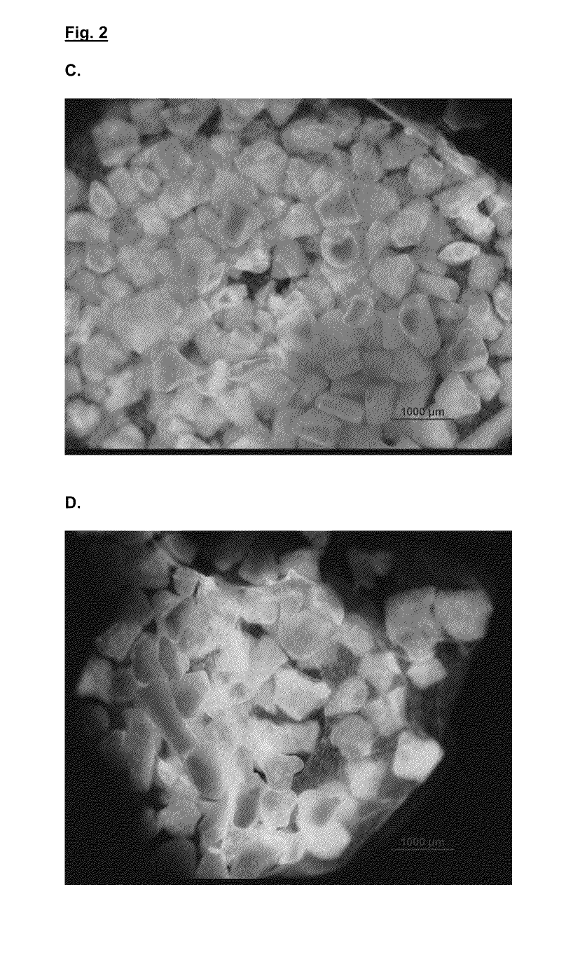

FIG. 2 shows the proof for the biological activity of the rhBMP-2 adsorbed on Algisorb (C and D) in the cell culture with MC3T3-E1 cells with:

A. Algisorb, negative control—no soluble rhBMP-2 in the medium);

B. Algisorb, positive control—addition of 50 nM soluble rhBMP-2 to the medium);

C. rhBMP-2 adsorbed to Algisorb (˜0.5 mg rhBMP-2 per g Algisorb)(kept moist);

D. rhBMP-2 adsorbed to Algisorb (˜0.5 mg rhBMP-2 per g Algisorb)(dried);

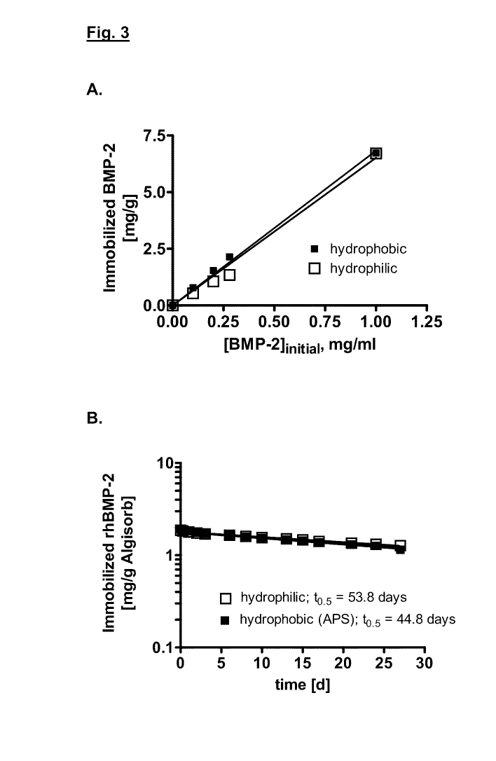

FIG. 3 A shows the hydrophilic and hydrophobic adsorptions of rhBMP-2; and

FIG. 3 B the release of rhBMP-2 (reaction of first order).

FIG. 1 shows an electron-microscopical photograph of the microstructured Algipore®, obtained from a limestone algae (Comp. AlgOss, Wien) having a substantially improved healing and resorption behavior compared to other porous hydroxyl apatites. The original CaCO₃ of the red limestone Cochlearia officinalis is replaced by hydroxyl apatite (HAP) (Algipore®) or tricalciumphosphate (TCP) (Algisorb®), maintaining the original microstructure [1].

As shown in FIG. 2, the proof of the biological activity of the rhBMP-2 adsorbed on Algisorb, was successful in the cell culture with MC3T3-E1 cells. Thereby, 5×10⁵ freshly trypsinated MC3T3-E1 cells were seed under sterile conditions on Algisorb-particles, being fixed on the bottom side with fibrin adhesive in the wells of a 48 micro titer plate, and incubated in Alpha-MEM medium (Gibco) with 10% FCS. 6-12 h later, the medium of the cells confluently grown on the plate is replaced by fresh alpha-MEM medium with 1% FCS, and the cells grew further on the control-Algisorb or the Algisorb (without functionalization with APS) with adsorbed BMP-2 for 6 days. After 6 days, the Algisorb-particles, populated with cells were washed with Dulbecco's phosphate buffer and fixed with 2% paraformaldehyde. The alkaline phosphatase (green fluorescent dye) was photographed with the phosphatase detection kit ELF-97 (Molecular Probes, Inc., Oregon, USA) using a fluorescence microscope (Nikon Eclipse E400, 10 Megapixel Camera, Nikon GmbH, Dusseldorf, Germany, excitation wave length 345 nm, emission wave length 530 nm) and determined.

As shown in FIG. 3, the following can be seen for the properties of the high density solid phase BMP-2 according to the invention. It can be calculated from the particle number of ˜1.3×10⁴ particle/g Algisorb at a load with rhBMP-2 of 6.7 mg/g, that 0.5 μg rhBMP-2 is bound per particle. This means that two particles (=1 μg) are sufficient to produce a significant bone induction in sheep experiments [2].

The improved properties of the Algipore are based, according to the findings of the inventors, on one side on the interconnecting pore system and the presence of isotropic (amorphous) hydroxyl apatite particles in contrast to the highly crystalline hydroxyl apatite in Bio-Oss (company Geistlich). The tricalciumphosphate containing version of the limestone algae, the Algisorb®, has thus a further improved resorption behavior, compared to Algipore, according to the present investigations.

The binding behavior is not only shown for Algipore and Algisorb but a similar behavior can be found for other hydroxyl apatites. The specifics for Algipore and Algisorb are that the bounded amounts are in the range of 1-2 mg/g and above. Such amounts are not known in the state of art until now. Using the inventive process, it is possible to obtain more than 7 mg BMP-2/g particle (2.8-4.4 mg/cm³) (high density solid phase BMP-2).

Information for the materials Algipore and Algisorb used in the invention can be taken from reference [1]. Thus, Algipore®: 98% hydroxyl apatite HA—monophasic, and Algisorb®: 80% tricalciumphosphate β-TCP, 19.3% HA, 0.7% calcite CaCO₃—bi/triphasic can be used according to the invention. For the latter one, all bi/triphasic versions of β-TCP and HA can be used with the same electron microscopic structure. The calcite is present in 0.3-0.7%.

Investigations of the inventors concerning the properties of the inventive high density solid phase BMP-2 have further revealed that it can be calculated from the particle number of ˜1.3×10⁴ particle/g Algisorb at a load of rhBMP-2 of 6.7 mg/g (FIG. 1), that 0.5 μg rhBMP-2 are bound per particle. This means that 2 particles (=1 μg) are sufficient to produce a significant bone induction in sheep experiments [2]. Thus, rhBMP-2 can be applied, rationally and without diffusion losses, in an inventive method in vivo and clinically.

For the production of the inventive high density solid phase BMP-2, it is worked in a range in which the BMP-2-content per volume unit is higher than the concentration which can be obtained in aqueous solutions. Preferably, buffer solutions of BMP are used according to the invention, preferably BMP-2, in a concentration of 0.1-1.5 mg/ml buffer solution, preferably 0.5-1.5 mg/ml buffer solution. A thus concentrated BMP-containing buffer solution is added to the particles in an amount so that the intended load in mg BMP per g particle is achieved. For example, 5 ml of buffer solution containing a 1 mg BMP per ml is added to 2 g particle if a load of 2.5 mg BMP per g particle is intended. If the test volume is increased in relation to the net weight by 4-times, 7 mg/g particle instead of 10 mg/g particle are bound.

Further, not yet finished investigations of the inventors show that higher amounts (from 4-5 mg BMP to 8-10 mg BMP/g particle) can be obtained. Accordingly, an amount (in ml) of a BMP-containing buffer solution which is predetermined in relation to the concentration can be used. It is therefore sufficient when applying the inventive particles loaded with bone growth factor if, during the application, just some grains of the BMP-HAP composition are applied, (for example together with an implant or during a bone augmentation of the maxillary antrum) to reproduce a bone induction. In vitro investigations of the inventors show that the BMP-2 bounded to HAP is biologically active. Further investigations of the inventors on the sheep are presently in progress.

Furthermore, it was found surprisingly by the inventors that tricalciumphosphate which is used in combination with hydroxyl apatite as bone replacement material and which constitutes approximately 80% in the above-mentioned Algisorb®, can undergo an activation reaction with the activation agents such as aminopropyl triethoxysilane, which leads to a further increase of the adsorption of BMP-2 to the particle surface by a factor of at least 2.

Accordingly, the inventors have shown that, per gram of a particulate material consisting of ˜80% TCP and ˜20% HAP (Algisorb), the same amount as for 98% HAP (Algipore) can be bound. This is more surprising as the skilled man would expect that, if HAP would be reacted only, additional 20% BMP only (=share of HAP in Algisorb) compared to Algipore with 98% HAP can be bound. It is assumed by the inventors that the additional 60-70% of bound BMP-2 is bound by a modified TCP. Accordingly, the present invention is also disclosing that the particulate material is activated by means of a treatment with an activation means before the adsorption of bone growth factors. The activation agent can be selected from the group of silanes, whereby the use of aminoalkyl alkoxysilanes such as aminopropyl triethoxysilane is preferred. Such activation treatment is usually effected by that the bone replacement materials (refer to table 1) are heated to boiling in 50 ml of a 5% (v/v) solution mixture of 3-Aminopropyl triethoxysilane (APS, Company Sigma-Aldrich, Taufkirchen) in dry toluene to reflux for 3.5 h under an inert gas atmosphere (Nitrogen 5.0) in heated glass equipment. After termination of the reaction, the samples are cooled down and separately washed 3-times each in 10 ml chloroform, 3-times in acetone and 3-times in methanol.

Thereafter, the so activated particulate material, especially consisting of TCP, HAP or mixtures thereof (see table 1) can be treated either in the acidic range in the range between ph 4 and 5, at pH 4.5 (20 mM Na-acetate-buffer, pH 4.5), or in weakly alkaline range between pH 9 and 11, preferred at pH 10 (125 mM borate, 0.066% SDS pH 10.0) buffered solution of the bone growth factor, preferred BMP-2 or BMP-7, over a period of at least 30 minutes (t_{1/2}=26 d), preferred for at least 4 hours (t_{1/2}=36 d) and particularly preferred 15 hours (t_{1/2}=43 d). For this, the particulate materials are washed, after chemical modification with aminopropyl triethoxysilane (APS), with water and subsequently transferred into small (2 ml) reaction vessels, in which 1.0 ml, respectively, of a BMP-2-solution either in 20 mM Na-Acetate-buffer pH 4.5 or 125 mM Borat/0.066% SDS-buffer, pH 10.0 is present. For the adsorption of rhBMP-2 to the materials, three different protein concentrations are used: 0.1, 0.2 and 0.3 mg/ml. The amount of immobilized BMP-2 is determined by using protein radioactively marked with ¹²⁵iodine.

In order to prevent the burst-phase, e.g. the excessive release of bone growth factor, which has not been adsorbed on the surface of the particles but simply remains thereon, the particles are preferably washed, after the adsorption step, preferably in three washing steps with 10-times volume of the particulate material in bone growth factor free buffer solution, (20 mM Na-acetate-buffer, pH 4.5 or 125 mM borate, 0.066 SDS pH 10.0) respectively. Thereafter, 5-times washing in PBS-buffer pH 7.4 (137 mM NaCl, 8.1 mM Na₂HPO₄, 2.7 mM KCl, 1.5 mM KH₂PO₄, pH 7.4) were carried out.

By providing the inventive high density solid phase BMP, it is possible to particularly apply rhBMP-2 rationally and without any diffusion losses in a new manner in vivo and clinically. It has been shown that high density solid phase BMP is storable after lyophilisation for several weeks without any loss of activity (according to FIG. 2). First investigations of the inventors show that the storability of the inventive high density solid phase BMP can be extended over a period of 1-2 years. Thereby, the biological activity of BMP is maintained, which can be attributed, according to the inventors, to the materials preferably used under sterile conditions.

### LITERATURE

[1] Spassova, E., Gintenreiter, S., Halwax, E., Moser, D., Schopper, C., & Ewers, R. (2007) Chemistry, Ultrastructure and Porosity of Monophasic and Biphasic Bone Forming Materials Derived from Marine Algae. Materialwiss. Werkstofftech., 38, 1027-1034.
[2] Lichtinger, T. K., Müller, R. T., Schürmann, N., Wiemann, M., Chatzinikoleidou, M., Rumpf, H. M., & Jennissen, H. P. (2001) Osseointegration of Titanium Implants by Addition of Recombinant Bone Morphogenetic Protein 2 (rhBMP-2). Materialwiss. Werkstofftech., 32, 937-941.

1. Process for preparing a particulate inorganic material loaded with growth factors, wherein said particulate material being selected from ceramic materials is treated with growth factors in aqueous buffered solution in the acidic range between pH 4 and 5 over a period of at least 30 minutes, then the particulate material is separated from the aqueous buffered solution and washed at least once with the same volume of the buffered solution free of growth factor.

2. Process according to claim 1, wherein said particulate material is dried after the washing step with the aqueous buffered solution free of growth factor.

3. Process according to claim 2, wherein said drying step comprises a lyophilisation.

4. Process according to claim 3, wherein said aqueous buffered solution has a pH-value of 4.3 to 4.7.

5. Process according to claim 4, wherein said particulate material has a particle size in the range of 10 to 500 μm and an interconnecting pore structure.

6. Process according to claim 5, wherein said particulate material is consisting of hydroxyl apatite, tricalcium phosphate, calcium carbonate, aluminum oxide or mixtures thereof.

7. Process according to claim 1, wherein a particulate material with a chemically activated surface is used.

8. Process according to claim 7, wherein said particulate material having a chemically activated surface is obtained by treating the particulate material with an activation agent.

9. Process according to claim 7, wherein BMP-2 or BMP-7 is used as growth factor.

10. Particulate material obtainable according to the process of claim 1.

11. Use of the particulate material of claim 10 for preparing a pharmaceutical composition for promoting bone growth.

12. Process according to claim 1, wherein said aqueous buffered solution has a pH-value of 4.3 to 4.7.

13. Process according to claim 1, wherein said aqueous buffered solution has a pH-value of 4.5.

14. Process according to claim 1, wherein said particulate material has a particle size in the range of 10 to 500 μm and an interconnecting pore structure.

15. Process according to claim 1, wherein said particulate material is consisting of hydroxyl apatite, tricalcium phosphate, calcium carbonate, aluminum oxide or mixtures thereof.

16. Process according to claim 1, wherein said particulate material having a chemically activated surface is obtained by treating the particulate material with an activation agent selected from the group of silanes.

17. Process according to claim 1, wherein at least one of BMP-2 and BMP-7 is used as growth factor
